(19) United States
(12) Patent Application Publication
(10) Pub. No.: US 2015/0164754 A1
(43) Pub. Date: Jun. 18, 2015

(54) ANTI-AGEING COMPOSITION CONTAINING DEHYDROGENATED ABIETIC ACID AS ACTIVE INGREDIENT

(71) Applicant: AMOREPACIFIC CORPORATION, Seoul (KR)

(72) Inventors: Young Gyu Kang, Yongin-si (KR); Jun Seong Park, Yongin-si (KR); Jin Sup Shim, Yongin-si (KR); Kyeong Hwan Hwang, Yongin-si (KR); Joon HO Park, Yongin-si (KR); Myeong Hun Yeom, Yongin-si (KR); Jun Cheol Cho, Yongin-si (KR)

(73) Assignee: Amorepacific Corporation

(21) Appl. No.: 14/403,333

(22) PCT Filed: May 15, 2013
(86) PCT No.: PCT/KR2013/004317

§ 371 Date: Nov. 24, 2014

(30) Foreign Application Priority Data
May 24, 2012 (KR) 1020120055099

(51) Int. Cl. A61K 8/36; A61Q 19/08

(52) U.S. Cl. CPC A61K 8/36; A61Q 19/08

(57) ABSTRACT

An objective of the present invention is to provide a skin ageing preventing composition that contains dehydrogenated abietic acid as an active ingredient and consequently has an outstanding effect in promoting skin cell and collagen production while not giving rise to any adverse skin reactions. Also, an objective of the present invention is to provide a method for preventing skin ageing by using the composition containing dehydrogenated abietic acid as an active ingredient.

### TECHNICAL FIELD

The present invention relates to an anti-aging composition containing dehydrogenated abietic acid as an active ingredient.

### BACKGROUND ART

The skin is a primary defense barrier of the human body, which functions to protect various organs in the body from changes in temperature and humidity, and external environmental stimuli such as UV and pollutants, and plays an important role in maintaining homeostasis in vivo. However, excessive physical and chemical stimuli from the external environment, stresses, nutritional deficiency and the like deteriorate the normal function of the skin and accelerate skin aging phenomena such as firmness loss, keratinization and wrinkle formation and the like. To prevent such phenomena and to keep the skin more healthy and elastic, there have been efforts to use cosmetics containing physiologically active substances obtained from various plants, microorganisms and the like, so as to maintain the intrinsic function of the skin and activate skin cells, thus effectively suppressing skin aging. However, cosmetic raw materials according to the prior art have various problems in that they mostly have insufficient effects or cause skin side effects.

Dehydrogenated abietic acid has been used mainly in coating compositions, and has also been used in compositions for fingernail or toenail protection, which are applied to the nail surface before application of nail polishes. However, the effect of dehydrogenated abietic acid on the stimulation of skin cell production and collagen production has not yet been reported.

### DISCLOSURE

### Technical Problem

Accordingly, the present inventors have found that dehydrogenated abietic acid has an excellent effect of stimulating skin cell production and collagen production without causing side effects on the skin. Therefore, it is an object of the present invention to provide a composition for preventing skin aging, which contains, as an active ingredient, dehydrogenated abietic acid that has an excellent effect of stimulating skin cell production and collagen production without causing side effects on the skin.

Another object of the present invention is to provide a method of preventing skin aging using a composition containing dehydrogenated abietic acid as an active ingredient.

### Technical Solution

In order to accomplish the above objects, in accordance with an embodiment of the preset invention, there is provided an anti-aging composition containing, as an active ingredient, dehydrogenated abietic acid represented by Formula 1 below, an isomer thereof, a precursor thereof, a hydrate thereof or a solvate thereof.

In accordance with another embodiment of the present invention, there is provided a method of preventing skin aging using a composition containing, as an active ingredient, dehydrogenated abietic acid represented by Formula 1 below, an isomer thereof, a precursor thereof, a hydrate thereof or a solvate thereof.

As used herein, the term “isomer” is particularly meant to include enantiomer, conformational isomers, positional isomers (particularly, tautomers) or geomeric isomers.

As used herein, the term “precursor” refers to a compound obtained by chemically modifying any compound to control the physical and chemical properties. It exhibits no physiological activity by itself, but can be converted to the original compound by chemical or enzymatic action in vivo after administration to exhibit its activity.

As used herein, the term “salt” refers to a “pharmaceutically acceptable salt”. The term “pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

As used herein, the term “pharmaceutically acceptable salts” means salts which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2,-ethanedisulfonic acid, 2-hydroxyethanesulfonic acid, benzenesulfonic acid 4-chlorobenzenesulfonic acid, 2-naphthalenesulfonic acid, 4-toluenesulfonic acid, camphorsulfonic acid, 4-methylbicyclo[2.2.2]oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or salts that are formed when an acidic proton present in a parent component is replaced.

The term “hydrate” means a “pharmaceutically acceptable hydrate”. The “hydrate” exists when the compound of the present invention contains water. The hydrate may contain one or more water molecules per molecule of the compound of the present invention. Illustrative non-limiting examples include monohydrates, dehydrates, trihydrates and tetrahydrates. The hydrate may contain at least one molecule of compound of the present invention per molecule of water. Illustrative non-limiting examples include semi-hydrates. In an embodiment, the water may be held in the crystal in various ways, and thus the water molecule may occupy lattice positions in the crystal, or may form a bond with a salt of the compound as described herein. The hydrate must be “acceptable” in the sense of not being deleterious to the recipient thereof.

The term “solvate” means a “pharmaceutically acceptable hydrate”. The term “solvate” means that the compound of the invention contains one or more pharmaceutically acceptable solvents. The solvate may contain one or more molecules of solvent per molecule of compound of the present invention, or may contain one or more molecules of compound of the invention per molecule of solvent. In one embodiment, the solvent may be held in the crystal in various ways, and thus the solvent molecule may occupy lattice positions in the crystal, or may form a bond with a salt of the compound as described herein.

Dehydrogenated abietic acid as described herein may be represented by the following Formula 1:

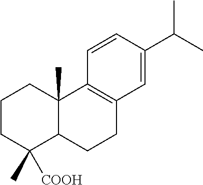 [SMILES: CC(C)c1ccc2c(c1)CCC1[C@](C)(C(=O)O)CCC[C@]21C]

In an embodiment of the present invention, the content of dehydrogenated abietic acid in the composition may be 0.0001-10 wt % based on the total weight of the composition. If the content of dehydrogenated abietic acid is less than 0.0001 wt %, the effect thereof will be insignificant, and if the content is more than 10 wt %, an increase in the content will not lead to a significant increase in the effect, and thus will not be cost-effective.

In an embodiment of the present invention, the anti-aging composition may be for reducing skin wrinkles.

In another embodiment of the present invention, the anti-aging composition may be for enhancing skin elasticity.

In still another embodiment of the present invention, the anti-aging composition may be for preventing aging by proliferating skin cells or fibroblasts.

In still another embodiment of the present invention, the anti-aging composition may be for preventing aging by stimulating collagen biosynthesis.

The present invention provides an anti-aging composition containing dehydrogenated abietic acid as an active ingredient.

In an embodiment of the present invention, there is provided a method of reducing skin wrinkles using the composition.

In another embodiment of the present invention, there is provided a method of enhancing skin elasticity using the composition.

In still another embodiment of the present invention, there is provided a method of proliferating skin cells or fibroblasts using the composition.

In yet another embodiment of the present invention, there is provided a method of stimulating collagen biosynthesis using the composition.

### Advantageous Effects

The anti-aging composition of the present invention, which contains dehydrogenated abietic acid as an active ingredient, exhibits an excellent effect of stimulating skin cell production and collagen biosynthesis without causing side effects on the skin. Due to this effect, the composition of the present invention will be useful as a skin external composition for anti-aging and wrinkle reduction.

### BEST MODE

Hereinafter, the present invention will be described in further detail.

The present invention provides an anti-aging composition containing dehydrogenated abietic acid as an active ingredient. The present invention also provides methods of reducing skin wrinkles, enhancing skin elasticity, and stimulating skin cell or fibroblast proliferation and collagen biosynthesis, by using the anti-aging composition containing dehydrogenated abietic acid as an active ingredient.

In an embodiment of the present invention, the content of dehydrogenated abietic acid in the composition may be 0.0001-10 wt % based on the total weight of the composition. If the content of dehydrogenated abietic acid is less than 0.0001 wt %, the effect thereof will be insignificant, and if the content is more than 10 wt %, an increase in the content will not lead to a significant increase in the effect, and thus will not be cost-effective.

The composition of the present invention, which contains dehydrogenated abietic acid as an active ingredient, may be, for example, a pharmaceutical composition or a cosmetic composition.

The anti-aging pharmaceutical composition may further comprise a pharmaceutical adjuvant such as a preservative, a stabilizer, a wetting agent or emulsifier, a salt for osmotic control and/or buffer or other therapeutically useful substance, and may be formulated into various oral or parenteral dosage forms according to methods known in the art. Parenteral dosage forms may be transdermal dosage forms, for example, lotion, ointment, gel, cream, patch or spray formulations, but are not limited thereto.

Determination on dosage of the active ingredient is within the knowledge of those skilled in the art. The daily dose of the active ingredient may vary depending on various factors, including the severity of the disease, the time of onset of the disease, the subject's age and health conditions, complications, etc. For an adult, the composition may generally be administered at a dose of 1 μg/kg to 200 mg/kg, preferably 50 μg/kg to 50 mg/kg, 1-3 times a day. The dose does not limit the scope of the present invention in any way.

The formulation of the anti-aging cosmetic composition is not specifically limited, and can be suitably selected depending on the intended use. For example, it may be one or more formulations selected from the group consisting of skin lotion, skin softer, skin toner, astringent, lotion, milk lotion, moisture lotion, nourishing lotion, massage cream, nourishing cream, moisture cream, hand cream, essence, nourishing essence, pack, soap, cleansing foam, cleansing lotion, cleansing cream, body lotion, and body cleanser, but is not limited thereto.

When the formulation of the present invention is paste, cream or gel, it may contain, as a carrier component, animal fiber, plant fiber, wax, paraffin, starch, tragacanth, cellulose derivatives, polyethylene glycol, silicone, bentonite, silica, talc, or zinc oxide.

When the formulation of the present invention is a power or spray formulation, it may contain, as a carrier component, lactose, talc, silica, aluminum hydroxide, calcium silicate or polyamide powder. Particularly, when the formulation is a spray formulation, it may further contain a propellant such as chlorofluorohydrocarbon, propane/butane or dimethyl ether.

When the formulation of the present invention is a solution or an emulsion, it may contain, as a carrier component, a solvent, a solubilizer or an emulsifier. Examples of this carrier component include water, ethanol, isopropanol, ethyl carbonate, ethyl acetate, benzyl alcohol, benzyl benzoate, propylene glycol, 1,3-butyl glycol oil, glycerol aliphatic ester, polyethylene glycol, or fatty acid ester of sorbitan.

When the formulation of the present invention is a suspension, it may contain, as a carrier component, a liquid diluent such as water, ethanol or propylene glycol, a suspending agent such as ethoxylated isostearyl alcohol, polyoxyethylene sorbitol ester or polyoxyethylene sorbitan ester, microcrystalline cellulose, aluminum metahydroxide, bentonite, agar, or tragacanth.

When the formulation of the present invention is a surfactant-containing cleanser, it may contain, as a carrier component, aliphatic alcohol sulfate, aliphatic alcohol ether sulfate, sulfosuccinic monoester, isethionate, imidazolinium derivatives, methyl taurate, sarcosinate, fatty acid amide ether sulfate, alkyl amidobetaine, aliphatic alcohol, fatty acid glyceride, fatty acid diethanolamide, vegetable oil, lanolin derivatives, or ethoxylated glycerol fatty acid ester.

The cosmetic composition may further contain, in addition to dehydrogenated abietic acid, a functional additive and other ingredients that are generally used in cosmetic compositions. The functional additive may be selected from the group consisting of water-soluble vitamins, oil-soluble vitamins, polypeptides, polysaccharides, sphingolipids, and seaweed extracts.

The cosmetic composition of the present invention may, if necessary, contain, in addition to the functional additive, components that are generally used in cosmetic compositions. Examples of such additional components include oil and fat components, moisturizers, emollients, surfactants, organic and inorganic pigments, organic powder, UV absorbers, preservatives, sterilizers, antioxidants, plant extracts, pH adjusters, alcohols, colorants, fragrance, blood circulation stimulants, skin coolers, deodorants, purified water, etc.

Furthermore, the present invention relates to a skin external formulation comprising the above-described anti-aging composition. The term “skin external formulation” is intended to include any formulations that are applied to the surface of the skin, including various cosmetic and pharmaceutical formulations. For example, the skin external formulation may be a formulation for preventing skin aging or reducing skin wrinkles, but is not specifically limited thereto.

In the present invention, it was found that the use of a composition containing dehydrogenated abietic acid has an excellent effect of preventing skin aging. As can be seen in the Test Examples to be described later, the anti-aging composition according to the present invention can function to reduce skin wrinkles by stimulating skin cell proliferation and collagen biosynthesis.

### MODE FOR INVENTION

Hereinafter, the present invention will be described in further detail with reference to examples. It is to be understood, however, that these examples are for illustrative purposes only and are not intended to limit the scope of the present invention.

### Test Example 1

### Effect on Fibroblast Proliferation

The effect of dehydrogenated abietic acid (purchased from Ramidus AB) of Formula 1 on wrinkle reduction was measured. In order to examine the effect of dehydrogenated abietic acid on skin cell proliferation that reduces wrinkles, human normal fibroblasts were seeded into a 96-well microplate at a density of 1×10⁴ cells/well, and incubated in DMEM (Dulbecco's Modified Eagle's Medium) medium in a CO₂ incubator at 36.5° C. for 24 hours. After incubation, in Example 1, the medium was replaced with a serum-free medium containing 10 μM of dehydrogenated abietic acid, and then the cells were further incubated for 24 hours. In Comparative Example 1, cells not treated with dehydrogenated abietic acid were used. Then, 10 μl of (3-(4,5-dimethyl-thiazol-2-yl)2,5-diphenyl tetrazolium (MTT, 5 mg/ml) was added to each well, which was then allowed to stand for 4 hours, followed by removal of the medium. Then, 100 μl of dimethyl sulfoxide solution was added to each well, and the absorbance at 570 nm was measured with a microplate reader.

Based on the absorbance measured as described above, the cell proliferation effect was calculated using the following equation. The results of the calculation are shown in Table 1 below.

Cell proliferation effect (%)=(absorbance of group treated with sample/absorbance of group not treated with sample)×100 Equation 1

TABLE 1

| | Concentration | | |
|---|---|---|---|
| | (μM) of | | Cell |
| | dehydrogenated | Absorbance | proliferation |
| | abietic acid | (570 nm) | effect (%) |
| Comparative | 0 | 0.300 | 0 |
| Example 1 | | | |
| Example 1 | 10 | 0.480 | 60.0 |

As can be seen from the results in Table 1 above, dehydrogenated abietic acid according to the present invention shows the effect of proliferating normal fibroblasts to reduce wrinkles.

### Test Example 2

### Effect on Stimulation of Collagen Biosynthesis

The effect of dehydrogenated abietic acid (purchased from Ramidus AB) of Formula 1 on the stimulation of collagen biosynthesis was measured. In order to examine the effect of dehydrogenated abietic acid on the stimulation of collagen biosynthesis, human normal fibroblasts were seeded into a 96-well microplate at a density of 1×10⁴ cells/well, and incubated in DMEM (Dulbecco's Modified Eagle's Medium) medium in a CO₂ incubator at 36.5° C. for 24 hours. After incubation, in Example 2, the medium was replaced with a serum-free medium containing 10 μM of dehydrogenated abietic acid, and then the cells were further incubated for 48 hours. In Comparative Example 2, cells not treated with dehydrogenated abietic acid were used. At 24 hours before completion of the incubation, 50 μg/ml of ascorbic acid was added to each well to stimulate collagen synthesis. After completion, each well was washed, and the medium was replaced with serum-free DMEM medium, after which the cells were further incubated for 24 hours. After incubation, the supernatant of each well was collected, and the amount of procollagen type-I C-peptide (PICP) in the supernatant was measured using a kit (Takara, Kyoto, Japan) to determine the amount of newly synthesized collagen. The amount of PICP that is the amount of collagen synthesized is expressed in units of ng/2×10⁴ cells in Table 2 below.

TABLE 2

| | Concentration (μM) of | Amount of collagen |
|---|---|---|
| | dehydrogenated abietic acid | synthesized (ng/2 × 10⁴) |
| Comparative | 0 | 130 |
| Example 2 | | |
| Example 2 | 10 | 220 |

As can be seen from the results in Table 2 above, the amount of collagen synthesized in Example 2 treated with dehydrogenated abietic acid was significantly larger than that in Comparative Example 2, suggesting that dehydrogenated abietic acid has the effect of stimulating collagen biosynthesis.

Hereinafter, formulation examples of the composition of the present invention, which contains dehydrogenated abietic acid as an active ingredient, will be described, but the composition of the present invention is not limited only to these examples.

### Formulation Example 1

### Lotion-Type Formulation

TABLE 3

| | Content (wt %) |
|---|---|
| Dehydrogenated abietic acid | 3.00 |
| L-ascorbic acid-2-magnesium sulfate | 1.00 |
| Water soluble collagen (1% aqueous | 1.00 |
| solution) | |
| Sodium citrate | 0.10 |
| Citric acid | 0.05 |
| Licorice extract | 0.20 |
| 1,3-butylene glycol | 3.00 |
| Purified water | Balance |

### Formulation Example 2

### Cream-Type Formulation

TABLE 4

| | Content (wt %) |
|---|---|
| Dehydrogenated abietic acid | 1.00 |
| Polyethylene glycol monostearate | 2.00 |
| Self-emulsifying glycerin | 5.00 |
| monostearate | |
| Cetyl alcohol | 4.00 |
| Squalene | 4.00 |
| Tri-2-ethylhexane glyceryl | 6.00 |
| Sphingoglycolipid | 1.00 |
| 1,3-butylene glycol | 7.00 |
| Purified water | Balance |

### Formulation Example 3

### Pack-Type Formulation

TABLE 5

| | Content (wt %) |
|---|---|
| Dehydrogenated abietic acid | 5.00 |
| Polyvinyl alcohol | 13.00 |
| L-ascorbic acid-2-magnesium sulfate | 1.00 |
| Lauroyl hydroxyproline | 1.00 |
| Water soluble collagen (1% aqueous | 2.00 |
| solution) | |
| 1,3-butylene glycol | 3.00 |
| Ethanol | 5.00 |
| Purified water | Balance |

### Formulation Example 4

### Essence-Type Formulation

TABLE 6

| | Content (wt %) |
|---|---|
| Dehydrogenated abietic acid | 2.00 |
| Hydroxyethylene cellulose (2% aqueous | 12.00 |
| solution) | |
| Xanthan gum (2% aqueous solution) | 2.00 |
| 1,3-butylene glycol | 6.00 |
| Concentrated glycerin | 4.00 |
| Sodium hyaluronate | 5.00 |
| Purified water | Balance |

### INDUSTRIAL APPLICABILITY

The use of an anti-aging composition containing dehydrogenated abietic acid as an active ingredient according to an embodiment of the present invention can exhibit the effect of stimulating skin cell proliferation and collagen biosynthesis without causing side effects on the skin. Due to this effect, the composition can provide a functional skin external composition for preventing skin aging, reducing wrinkles and enhancing skin elasticity, etc. In addition, the present invention may provide a method of reducing skin wrinkles, enhancing skin elasticity, and stimulating skin cell or fibroblast proliferation and collagen biosynthesis, by using this skin external composition.

1. An anti-aging composition containing dehydrogenated abietic acid as an active ingredient.

2. The anti-aging composition of claim 1, wherein the dehydrogenated abietic acid is contained in an amount of 0.0001-10 wt % based on the total weight of the composition.

3. The anti-aging composition of claim 1, which is used for reducing skin wrinkles.

4. The anti-aging composition of claim 1, which is used for enhancing skin elasticity.

5. The anti-aging composition of claim 1, which is used for preventing aging by stimulating proliferation of skin cells or fibroblasts.

6. The anti-aging composition of claim 1, which is used for preventing aging by stimulating collagen biosynthesis.

7. The anti-aging composition of claim 1, which is a pharmaceutical composition.

8. The anti-aging composition of claim 1, which is a cosmetic composition.

9. A method of reducing skin wrinkles by using the composition of claim 1.

10. A method of enhancing skin elasticity by using the composition of claim 1.

11. A method of proliferating skin cells or fibroblasts by using the composition of claim 1.

12. A method of stimulating collagen biosynthesis by using the composition of claim 1.
